(19) United States
(12) Patent Application Publication
(10) Pub. No.: US 2007/0218143 A1
(43) Pub. Date: Sep. 20, 2007

(54) Topical preparation

(76) Inventor: Mok Thoong Marvin Chong, Portage, MI (US)

Correspondence Address: JOSEPH KOORKIN ANDONIAN, 5137 RIDGEBROOK DRIVE, PORTAGE, MI, 49002, US

(21) Appl. No.: 11/384,972

(22) Filed: Mar. 20, 2006

(51) Int. Cl. A61K 36/534; A61K 35/60; A61K 35/64; A61K 31/125

(52) U.S. Cl. 424/523; 424/539; 424/747; 514/692

(57) ABSTRACT

A pharmaceutically elegant, rapidly penetrating, therapeutically useful topical combination of thymol, cod liver oil, camphor, peppermint oil and beeswax and the method for the preparation thereof.

This invention relates to a pharmaceutical method and the topical formulation produced thereby. More particularly it relates to a pharmaceutical method for producing a topical analgesic ointment that promotes healing and the ointment produced thereby.

### BACKGROUND OF THE INVENTION

A large body of literature verifies the topical use, at least individually, of thymol, peppermint oil, camphor, beeswax and cod liver oil either as therapeutically active ingredients or as formulation adjuvants for topical preparations. For example the Eleventh Edition of The Merck Index (1986) describes thymol as a topical antiseptic; peppermint oil as a oral flavoring agent; camphor as a topical anti-infective, antipruritic, and counterirritant; beeswax as a pharmaceutic aid in ointments and plasters; and cod liver oil as useful locally to promote healing in animals. The benefits of cod liver oil are summarized on the Web to include improvements in blood flow, blood pressure, coronary heart disease, bones and teeth, vision, healthy skin and mucous membranes, pregnancy and arthritis, primarily due to its content of vitamins A and D and omega 3 fatty acids. Cod liver oil is also an ingredient in a skin protectant ointment sold under the registered trademark DESITIN.

The patent literature also contains many disclosures of the individual therapeutically active ingredients of the present invention as well as sub combinations of those ingredients. Key word searches in the U.S. Patent Office reveal that cod liver oil, thymol and camphor are disclosed together in 40 different patents; cod liver oil and thymol in 54 patents; cod liver oil and camphor in 159 patents; and camphor and thymol in 540 patents. However these disclosures do not necessarily disclose actual combinations of these ingredients. Moreover the addition of peppermint oil and beeswax are critical to the present invention to provide a pharmaceutically elegant as well as a therapeutically effective combination especially in the quantities used.

Key word searches in the Patent Office and on the Web have not disclosed the combination of all five ingredients in a single preparation albeit four patents have been found that disclose all five ingredients. U.S. Pat. Nos. 5,145,675; 5,357,636; 6,008,246 and 6,673,756 all disclose the five ingredients of the present invention in long lists of other ingredients but not in the same specific combination. The lists are so long and the uses so varied that it would be inventive simply to pick out and combine the ingredients for the purposes of the present invention.

The therapeutically active ingredients (camphor, cod liver oil and thymol) have been associated, but apparently not physically combined, in the literature. The individual ingredients and sub-combinations of the individual ingredients are well known to be useful topical ingredients. For example, in a reprint found in the 1976 FDA CONSUMER (HHS Publication No. 77-3029), the three ingredients are associated in a list of Antitussives while camphor and thymol are associated in a list of nasal decongestants. The three ingredients are also included in a list of 90 active ingredients investigated by an FDA Advisory Panel called on by the FDA to study the effectiveness of non-prescription cold remedies. The Panel concluded that none of the products including any of the 90 ingredients would prevent, cure or even shorten the course of the common cold. Camphor and peppermint oil have been used together in deodorant compositions as refreshing agents (see U.S. Pat. No. 6,497,862); in a sun screen patch as counterirritants (see U.S. Pat. No. 6,924,410); and as gel forming agents and in plasters for topical use (see U.S. Pat. No. 5,886,011); Peppermint oil and thymol are also listed together as additives for use in plasters (see U.S. Pat. No. 5,886,011).

The literature contains considerable evidence about the therapeutic properties of cod liver oil, perhaps the most important ingredient in the present combination. Terkelsen el al, Scand J. Plast Reconstr Surg Hand Surg 34 (1): 15-20, 2000 concluded that “Topical application of cod liver oil ointment accelerates wound healing: an experimental study in wounds in the ears of hairless mice.” More specifically, Terkelsen et al conclude that “topical 25% cod liver oil ointment significantly accelerated both the epithelial and the vascular component of healing compared with saline. Vitamin A seems to have an important role in accelerating wound healing and could be the active component in cod liver oil.” Gruenwald J. et al, Adv. Ther. 19 (2): 101-107, 2002 conducted clinical studies on the effect of cod liver oil on symptoms of rheumatoid arthritis and concluded that orally administered cod liver oil can be recommended for the treatment of rheumatoid arthritis. Curtis C L et al, Drug Discov. Today 9 (7): 336, 2004 reviewed the literature and found that several clinical trials, animal feeding experiments and in vitro models that mimic cartilage destruction in arthritic disease demonstrated the efficacy of cod liver oil in the diet as an alternative treatment for pathological manifestations of arthritic disease.

The method and combination of the present invention are not only new but also uniquely solve the problem. of combining all five ingredients to produce a pharmaceutically elegant topical ointment useful as an analgesic and enhanced healing agent with rapid penetration. The combination of those ingredients with those properties are neither disclosed nor suggested by the prior art.

### SUMMARY OF THE INVENTION

The present invention comprises the combination of thymol, camphor and cod liver oil in a topical preparation including peppermint oil and beeswax. The method of the present invention comprises mixing thymol, peppermint oil and camphor in one container, melting cod liver oil and beeswax together in another container at a temperature below the boiling point, mixing the thymol-peppermint oil-camphor preparation with the hot cod liver oil-beeswax preparation, and pouring the resulting smooth yellowish ointment into dispensing units before it cools. The individual ingredients of the combination should equal USP standards. The concentration of cod liver oil should preferably exceed 50 percent by weight of the total combination and preferably ranges from about 55 to about 60 percent by weight of the total weight of the combination. The thymol, peppermint oil and camphor are preferably present in substantially equal amounts by weight and can vary from about 10 to about 15 percent of the total weight of the combination. The concentration of the beeswax is not critical except that it should be present in a sufficient amount to produce a smooth pharmaceutically elegant ointment, the amount generally varying from about 8 to about 10 percent by weight of the combination.

### DETAILED DESCRIPTION

A preferred embodiment of the method of the present invention employs the following ingredients in multiples of the following amounts:

| Ingredients | Amounts |
|---|---|
| Thymol | 20 gm |
| Peppermint oil | 20 gm |
| Camphor | 20 gm |
| Beeswax | 15 gm |
| Cod liver oil | 100 gm |

All ingredients are USP grade. The beeswax can be either white or yellow. The thymol, peppermint oil and camphor are mixed in a stainless steel container until they blend and turn white in color. The cod liver oil is poured over the beeswax in another stainless steel container and the combined ingredients are heated until they melt being careful not to exceed 60 degrees centigrade or a temperature below the boiling point of beeswax (62-65 degrees). All five ingredients are mixed together and blended using a mixer until they form a yellowish homogenous liquid. The resulting hot ointment mixture is poured into dispensing units and packaged. The cooled product forms a smooth yellowish pharmaceutically elegant ointment useful for the temporary relief of minor aches and pains associated with arthritis, simple backache, muscle soreness, tendonitis strains and sprains.

A commercial batch of the ointment would approximate 120 kilograms and the dispensing units would be glass jars large enough to hold 60 grams or about 6 ounces of ointment. For such a batch, 68.4 kilograms of cod liver oil, 13.2 kilograms of camphor, peppermint oil and thymol, and 10.8 kilograms of beeswax would be employed. The method of manufacture would be the same as indicated above.

In clinical experiments, the ointment of the present invention has been shown to promote faster healing of skin, muscles, cartilage and possibly bone probably because it rapidly penetrates and nourishes the skin. It also relieves aches and pains in the process of healing. The product is stable and has a long shelf life.

The foregoing provides both a general description and a specific description of the preferred embodiment of the invention. It should be understood that various substitutions, variations and modifications can be made by those skilled in the art without departing from spirit and scope of the invention as further delineated in the following claims.

I claim:

1. The method of preparing an elegant pharmaceutically useful topical ointment comprising the following steps
(a) mixing thymol, peppermint oil and camphor together until they blend,
(b) separately pouring cod liver oil over beeswax, heating at a temperature below the boiling point and mixing until the cod liver oil blends with the beeswax,
(c) combining step (a) and step (b) products using a mixer until a homogenous yellowish liquid is formed and
(d) filling suitable dispensing containers with the liquid resulting from step (c) before it solidifies.

2. The ointment produced by the method of claim 1 wherein the thymol, peppermint oil and camphor individually comprise from about 10 to about 15 percent by weight of the total ointment, the cod liver oil comprises from about 55 to about 60 percent by weight of the total ointment and beeswax comprises from about 8 to 10 percent by weight of the total ointment.

3. A pharmaceutically elegant, rapidly penetrating, therapeutically useful topical ointment comprising thymol, cod liver oil, camphor, peppermint oil and beeswax.

4. The ointment of claim 4 wherein the cod liver oil is present at a concentration by weight of about 55 to about 60 percent; the thymol, camphor and peppermint oil are individually present at a concentration by weight of about 10 to about 15 percent; and the beeswax is present at a concentration by weight of about 8 to about 10 percent of the total weight of the ointment.
